(19) United States
(12) Patent Application Publication
(10) Pub. No.: US 2026/0115049 A1
(43) Pub. Date: Apr. 30, 2026

(54) DEVICE FOR CONTROLLING MOISTURE CONTENT WITHIN A WOUND AND MONITORING THE WOUND HEALING PROCESS

(71) Applicant: Biodaptive Advanced Materials, LLC, Ronkonkoma, NY (US)

(72) Inventors: Daniel RIVEST, Charlotte, VT (US); Matthew Robert HENRY, State College, PA (US)

(21) Appl. No.: 19/371,723

(22) Filed: Oct. 28, 2025

Related U.S. Application Data
(60) Provisional application No. 63/713,295, filed on Oct. 29, 2024.

(51) Int. Cl. A61F 13/00; A61F 13/0206; A61F 13/0246; A61L 15/26

(52) U.S. Cl. CPC A61F 13/00051; A61F 13/0206; A61F 13/0246; A61L 15/26; A61F 2013/0074; A61F 2013/00744; A61F 2013/00944

(57) ABSTRACT

A bandage modulates the moisture level of a healing wound and includes sensors for detecting, collecting, and reporting data for various wound variables. The bandage may include an antimicrobial agent to reduce the bioburden of microorganisms within the bandage.

The present application claims benefit of and priority to U.S. Provisional Patent Application Ser. No. 63/713,295 filed Oct. 29, 2024 entitled DEVICE FOR CONTROLLING MOISTURE CONTENT WITHIN A WOUND AND MONITORING THE WOUND HEALING PROCESS, the entire content of which is hereby incorporated by reference herein.

### BACKGROUND

### Field of the Disclosure

The present invention relates to a bandage or dressing that can modulate the moisture content of a healing wound environment through the use of a compact, flexible fabric-like pump integrated into the bandage/dressing. The dressing includes an array of embedded sensors for collecting and reporting information pertaining to various parameters known to play a pivotal role in the wound healing process (i.e., pH, temperature, resistance, humidity, etc.) and/or an antimicrobial agent.

### Related Art

It has long been recognized that a moist environment enhances wound healing process and reduces scar formation, however, in order to enhance wound healing, a preferred moisture level should be maintained. Too little moisture will impede the re-epithelialization process while excess moisture often results in wound infections and can also lead to maceration of the periwound tissue. Despite recognizing the role moisture content within a wound environment plays in the wound healing process, conventional dressings/bandages do not actively modulate the moisture level at a wound site. Conventionally, clinicians relied on trying to match a wound dressing to the specific wound characteristics (e.g., exudate level). These dressings would then “passively” transport excess wound exudate by absorbing the liquid and transferring it to a hygroscopic, absorbent layer within the dressing where it was captured and retained until the dressing was removed. Since the 1990s, vacuum-assisted closure (VAC) or negative pressure wound therapy (NPWT) has provides active removal of excess exudate from wounds that exhibited high levels of exudate. While these types of systems can provide great benefits, there are a number of limitations that restrict their practical use including lack of portability, discomfort and high cost.

Accordingly, it would be beneficial to provide a dressing that is capable of actively controlling the moisture level of a wound to provide an ideal wound healing environment as well as supplying relevant and timely information about the wound as it heals.

### SUMMARY

It is an object of the present disclosure to provide a bandage or dressing that modulates moisture content of a healing wound.

In embodiments, the bandage or dressing may contain one or more sensors for the purposes of detecting, collecting and reporting relevant information regarding the wound.

A bandage including: a silicone contact layer positioned adjacent a wound and a user's skin when the bandage is in place; an electroosmotic pump mounted above the silicone contact layer, away from the wound, and operable to selectively draw fluid through the silicone contact layer; an absorbent layer positioned above the electroosmotic pump and in fluid communication therewith such that the absorbent layer absorbs fluid that is drawn using the electroosmotic pump; a flexible top layer provided above the absorbent layer; and an electronic module electrically connected to the electroosmotic pump to selectively activate the electroosmotic pump to draw fluid away from the wound and control a moisture level at the wound.

In embodiments, the silicone contact layer includes a plurality of openings formed therethrough and configure to allow fluid to pass from the wound to the electroosmotic pump.

In embodiments, the bandage includes an adhesive layer provided between a bottom surface of the silicone contact layer and configured to secure the silicone contact layer to the user's skin.

In embodiments, the electroosmotic pump is in fluid communication with the wound via the plurality of openings in the silicone contact layer.

In embodiments, the electroosmotic pump includes: an anode positioned adjacent a top surface of the silicone contact layer; a porous membrane positioned above the anode; and a cathode provided above the porous membrane and adjacent to the absorbent, wherein, when a voltage is applied between the anode and the cathode, liquid is drawn upward from the wound, through the silicone contact layer, through the porous membrane and into the absorbent layer.

In embodiments, the electroosmotic pump includes a first bonding layer positioned between the anode and the porous membrane and a second bonding layer positioned between the porous membrane and the cathode.

In embodiments, the electronics module is operably connected to the anode and the cathode and includes a control circuit to apply a voltage between the anode and the cathode.

In embodiments, the control circuit varies the voltage applied between the anode and the cathode to maintain desired moisture level in the wound.

In embodiments, the bandage includes a sensor provided adjacent the wound and operably connected to the electronics module to provide wound information associated with the wound to control operation of the electroosmotic pump based on the wound information.

In embodiments, the electronics module includes memory and the wound information is stored in the memory.

In embodiments, the electronics module includes a transceiver configured to send and receive information wirelessly and the wound information is sent to a remote electronic device.

In embodiments, control information is received by the transceiver and used to control operation of the electroosmotic pump.

In embodiments, the bandage includes a first cathode electrically connected to the electronic module, a first anode electrically connected to the electronic module, wherein the first cathode and the first anode are positioned in contact with the user's skin and apply a voltage to the wound.

A bandage in accordance with another embodiment of the present disclosure includes: a silicone contact layer positioned adjacent a wound and a user's skin when the bandage is in place; a foam layer positioned above the silicone contact layer such that fluid from the wound passes therethrough; a nonwoven layer positioned above the foam layer, wherein exudate passes from the foam later through the nonwoven layer and the nonwoven layer filters debris from the exudate; an electroosmotic pump mounted above the nonwoven layer, away from the wound, and operable to selectively draw fluid through the foam layer, the nonwoven layer and the silicone contact layer; an absorbent layer positioned above the electroosmotic pump and in fluid communication therewith such that the absorbent layer absorbs fluid that is drawn using the electroosmotic pump; a flexible top layer provided above the absorbent layer; and an electronic module electrically connected to the electroosmotic pump to selectively activate the electroosmotic pump to draw fluid away from the wound and control a moisture level at the wound.

In embodiments, the silicone contact layer includes a plurality of openings formed therethrough and configure to allow fluid to pass from the wound to the electroosmotic pump.

In embodiments, the bandage includes an adhesive layer provided between a bottom surface of the silicone contact layer and configured to secure the silicone contact layer to the user's skin.

In embodiments, the electroosmotic pump is in fluid communication with the wound via the plurality of openings in the silicone contact layer.

In embodiments, the electronics module is operably connected to the electroosmotic pump and includes a control circuit to apply a voltage.

In embodiments, the control circuit varies the voltage to the electroosmotic pump to maintain a desired moisture level in the wound.

In embodiments, the electronics module includes a timer and the electroosmotic pump is controlled based in timing information.

In embodiments, the bandage incudes a sensor positioned at the electroosmotic pump and operable connected to the electronics module, wherein the sensor provides moisture information at the electroosmotic pump and the electroosmotic pump is controlled based on a moisture level at the electroosmotic pump.

In embodiments, the bandage includes a first cathode electrically connected to the electronic module and a first anode electrically connected to the electronic module, wherein the first cathode and the first anode are positioned in contact with the user's skin and apply a voltage to the wound.

### BRIEF DESCRIPTION OF THE DRAWINGS

The above and related objects, features and advantages of the present disclosure will be more fully understood by reference to the following, detailed description of the preferred, albeit illustrative, embodiments of the present invention when taken in conjunction with the accompanying figures, wherein:

FIG. 1 illustrates a schematic cross-sectional view of a bandage including a device for modulating moisture level at the site of a wound and for detecting, collecting and reporting information regarding various wound variables in accordance with an embodiment of the present disclosure;

FIG. 2 illustrates an exploded view of a bandage including a device for modulating moisture level of a wound in accordance with another embodiment of the present disclosure;

FIG. 3a illustrates an example of a cathode and an anode connected to an electronics module to apply voltage to a wound showing a first orientation of the cathode and the anode in accordance with another embodiment of the present disclosure;

FIG. 3b illustrates an example of a cathode and an anode connected to an electronics module to apply voltage to a wound showing another orientation of the cathode and the anode in accordance with another embodiment of the present disclosure;

FIG. 3c illustrates an example of a cathode and multiple anodes connected to an electronics module to apply voltage to a wound showing another orientation of the cathode and the anodes in accordance with another embodiment of the present disclosure; and

FIG. 3d illustrates an example of a cathode and multiple anodes connected to an electronics module to apply voltage to a wound showing another orientation of the cathode and the anodes in accordance with another embodiment of the present disclosure.

### DETAILED DESCRIPTION OF THE EXEMPLARY EMBODIMENTS

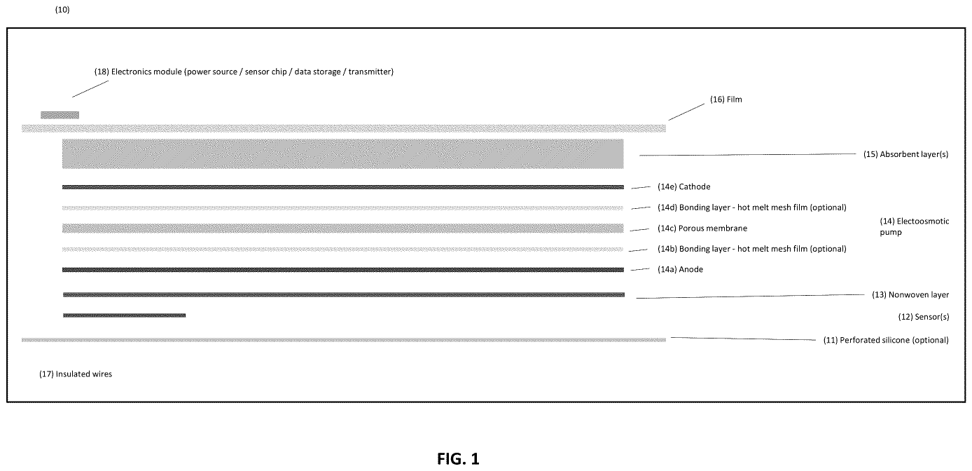

A bandage 10 including a device 1 for modulating moisture level at the site of a wound is illustrated in FIG. 1. In embodiments, the bandage 10 includes an embedded electroosmotic pump 14. In embodiments, the electroosmotic pump 14 may include an anode 14a. In embodiments, the anode 14a may be a water permeable, conductive material. In embodiments, the anode 14a may be a native material or may be implemented using a treatment/coating applied to a material to alter the conductive properties of the material. In embodiments, the anode 14a may be made of one or more conductive materials, such as carbon fibers, metal-plated nonwoven, graphene or poly(3,4-ethylenedioxythiophene) polystyrene sulfonate, to name a few.

In embodiments, the electroosmotic pump 14 also includes a cathode 14e. In embodiments, the cathode 14e may be a water permeable, conductive material. In embodiments, the cathode 14e may be a native material or may be implemented using a treatment/coating applied to a material to alter the conductive properties of the material in a manner similar to that discussed above with respect to the anode 14a. In embodiments, the cathode 14e may be made of one or more conductive materials, such as carbon fibers, metal-plated nonwoven, graphene or poly(3,4-ethylenedioxythiophene) polystyrene sulfonate, to name a few.

In embodiments, a porous membrane 14c may be positioned between the anode 14a and cathode 14e such that they are separated. In embodiment, the porous membrane 14c may be a native material or implemented using a treatment/coating applied to alter the surface chemistry of the material. In embodiments, the porous membrane 14c may be made of one or more porous materials, such as glass fiber membranes, quartz fiber membrane, track-etched polycarbonate membranes, to name a few.

In embodiments, the anode 14a, the porous membrane 14c, and the cathode 14e may be adhered to one another using bonding layers 14b and 14d. In embodiments, the bonding layers 14b and 14d may be made of one or more bonding materials, such as adhesive film, hot melt resin, thermosetting resin, stamped rubber film, to name a few.

In embodiments, one or more absorbent layer(s) 15 may be positioned on the top, non-patient contacting side of the electroosmotic pump 14 in contact with the cathode 14e. As fluid is transported away from the wound site by electroosmotic pump 14, it is transferred into, and retained by, the absorbent layer(s) 15. In embodiments, the absorbent layer(s) 15 may be made of one or more absorbent materials, such as medical grade polyurethane foam, super absorbent fiber, super absorbent polymer, hydrocolloid, carboxymethyl cellulose, alginate, spunbound polypropylene, or meltblown polypropylene, to name a few.

In embodiments, one or more sensors 12 may be provided on or near the wound facing side of the bandage 10. In embodiments, the one or more sensors 12 may be separated from the wound by a medical grade perforated silicone film or layer 11. In embodiments, a nonwoven layer 13 separates sensor 12 from electroosmotic pump 14. In embodiments, the sensor(s) 12 may be implemented using a metal-oxide semiconductor sensor chip, a working electrode, a counter electrode, and a reference electrode. In embodiment, the sensor(s) 12 may be powered by an electronics module 18. In embodiments, the electronic module 18 may power the sensors 12 via a wired connection or wirelessly. In embodiments, sensor chips may be integrated into electronics module 18 located on the non-patient contacting side of the bandage 10 attached to the polyurethane film 16, for example. In embodiment, data may be communicated from the working electrode, a counter electrode and a reference electrode of at least one sensor 12 to the sensor chip on the electronic module 18 either wirelessly or by insulated wires 17, for example. In embodiments, the layers of the bandage 10 may include contact areas that may be aligned such that they are in contact with each other throughout the bandage. In embodiments, these contact areas may be used to provide power to the anode 14a and cathode 14e and/or to the sensors 12. In embodiments, these contact areas may also be used to provide information from the sensors 12 to the electronic module 18. In embodiments, data collected by the at least one sensor 12 may be stored locally in the electronics module 18 and/or may be transmitted wirelessly to a computer, or portable electronic device, for viewing and long-term storage of data. In embodiments, the data collected may be transmitted to and stored in or processed by a remote server that may be associated with a clinician, patient or third party. In embodiments, the data collected may be transmitted wirelessly or via a wired connection to the cloud or to or via any communication network, for example, the Internet.

In embodiments, the sensor(s) 12 may be used to provide moisture information indicative of moisture at or near the wound. For example, the sensor 12 may be positioned above the silicone layer to provide moisture information indicative of moisture from the wound. In embodiments, the sensor 12 may provide moisture information indicative of a moisture level elsewhere in the bandage, for example, at the absorbent layer 15, for example. In embodiments, the sensor 12 may be used to provide other information associated with the wound or the bandage. In embodiment, the sensor 12, may provide temperature information indicative of a temperature at the wound or elsewhere, which may be indicative of wound healing or possible infection. In embodiments, the sensor 12 may be used to gather other information about the wound or bandage, for example, presence of bacteria, exposure to light, levels of gas, to name a few.

In embodiments, excess exudate from the wound may be absorbed into the nonwoven layer 13 and/or anode 14a. In embodiments, the surface of porous membrane 14c may be charged when in contact with an aqueous-based liquid and a diffuse layer of ions forms to screen the surface charge. In embodiments, a DC voltage bias may be applied between anode 14a and cathode 14e. In embodiments, the electric field created by the DC bias causes the diffuse layer of ions at the surface of the pores within the porous membrane 14c to migrate toward the oppositely charged electrode. In the pores, the migration of the diffuse layer toward the oppositely charged electrode causes the bulk fluid within the channel to flow through viscous drag. In embodiments, the rate of this flow can be modulated by the magnitude of the DC voltage bias applied. In embodiments, the wound fluid that is transferred from the anode 14a to the cathode 14e may subsequently be absorbed, and retained, by absorbent layer(s) 15 until the dressing is removed from the wound. In embodiments, the DC voltage bias may be using the electronic module 18. In embodiments, control signals may be generated to maintain a desired moisture level at the wound site. In embodiments, the electronic module 18 may provide the DC voltage to the anode 14a and cathode 14e.

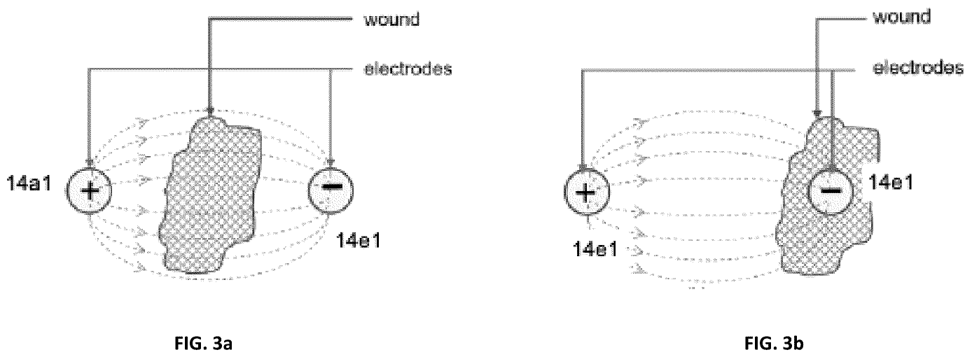

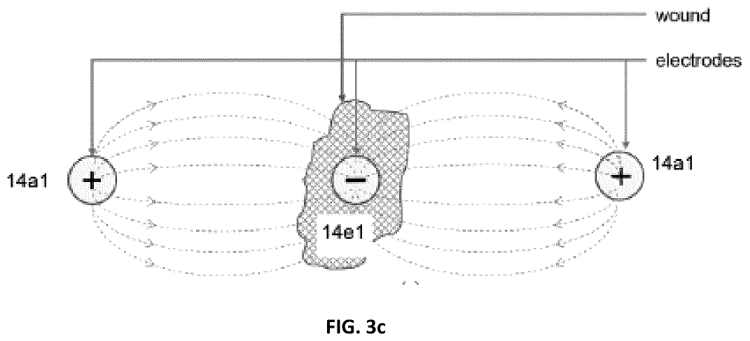

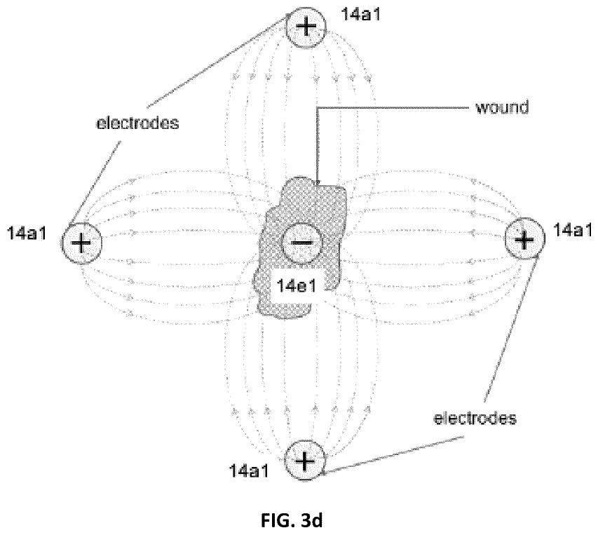

In embodiments, the DC bias voltage may be used to provide electrical stimulation to the wound itself. In embodiments, additional electrodes may be provided to apply a voltage to the wound. In embodiments, at least one second cathode 14e1 and at least one second anode 14a1 may be positioned beneath the perforated silicone layer 11 such that they are in direct contact with the wound bed as generally illustrated in FIG. 3a. In embodiments, the cathode 14e1 may be located at a center of the perforated silicone layer 11 of the dressing, aligned with a center of the wound. In embodiments, the anode 14a1 may be positioned along an outer edge or perimeter of the perforated silicone layer 11 over the periwound skin, which is the healthy tissue immediately surrounding the wound as can be seen in FIG. 3b, for example. In embodiments, the cathode 14e1 is positioned in the center of the wound and multiple anodes 14a1 are positioned around the periphery of the layer 11 over the periwound skin as shown in FIG. 3c. In embodiments, the cathode 14e1 is positioned in the center of the wound and multiple anodes 14a1 are positioned surrounding the periphery of the layer 11 over the periwound skin as shown in FIG. 3d. As an alternative to using multiple anodes, a single continuous thin circular electrode may be placed around the perimeter of the perforated silicone layer 11 which will serve as the anode, while a smaller central electrode serves as the cathode, for example. This configuration creates a “bull's-eye” pattern which simplifies the design while potentially maintaining effective current distribution across the entire wound area. In embodiments, the electrodes 14e1, 14a1 may be connected to the electronics module (18). In embodiments, electrical stimulation may speed wound healing by increasing capillary density and profusion, improving wound oxygenation and encouraging granulation and fibroblast activity. In embodiments, the anode 14a1 and cathode 14e1 provided in contact with the user's skin may be in addition to those provided in the pump 14.

In embodiments, antimicrobial agent(s) may be coated on, or embedded into, any of the various components of the bandage 10. In embodiments, the antimicrobial agent(s) reduce the bioburden of live microorganisms that are transferred into the dressing from the wound exudate. In embodiments, the antimicrobial agent(s) may be provided on or embedded in the anode 14a, cathode 14e, or absorbent layer(s) 15. In embodiments, antimicrobial agents may be made of or include one or more of polyhexamethylene biguanide, chlorhexidine gluconate, silver metal/nanoparticle, silver ions, zinc metal/nanoparticle, zinc ions, copper metal/nanoparticle, copper ions, to name a few. In embodiments, one or more of the anode 14a and the cathode 14e may be made of a conducting antimicrobial material, for example, a graphene silver membrane.

The bandage 10 provides many advantages including: (1) offering a compact, flexible, and cost-effective wound dressing that can the control moisture level within the wound environment to aid in the wound healing process and (2) supply information pertaining to the wound to clinicians and/or patients that may allow for the monitoring of the wound and/or guide the treatment plan. In embodiments, the information pertaining to the wound may include a moisture level at the wound, pH level, temperature or electrical resistance. In embodiments, the information pertaining to the wound may be provided to the electronic module 18 and used to control operation of the electroosmotic pump 14 and maintain a desired moisture level at the wound. In embodiments, the wound information may be stored and processed at the electronic module 18 or may be sent to a clinician or patient computer system or mobile electronic device. In embodiments, the electronic module 18 may include a transmitter/receiver to send and receive data with a computer system or mobile electronic device associated with a clinician or a patient. The electronic module 18 may transmit the moisture information to a computer system, mobile electronic device network or the cloud. In embodiments, the sensors 12 may include a transmitter/receiver that may be used to send wound information associated with the wound directly to a computer system or mobile electronic device, network or the cloud associated with the clinician or the patient. In embodiments, information may be sent to a remote server, which may be associated with the clinician or the patient or with a third party.

In embodiments, the electronic module 18 may include one or more processors to process the information provided from the sensors 12 and may include at least one memory operably connected thereto to store the information as well as processor executable code that when executed by the at least one processor processes the information provided by the sensors to provide suitable control signals to control the electroosmotic pump 14 to maintain a desired moisture level, address pH levels, apply voltage to the wound or determine electrical resistance of the wound. Electrical resistance and PH levels at the wound may provide indications of how the wound is healing, which may be used to determine whether to vary the voltage provided to the pump 14 to adjust moisture levels or may be used to provide voltage to the wound. In embodiments, the control signals to the electroosmotic pumps may be based on instructions provided from a clinician or patient via a computer system or mobile electronic device.

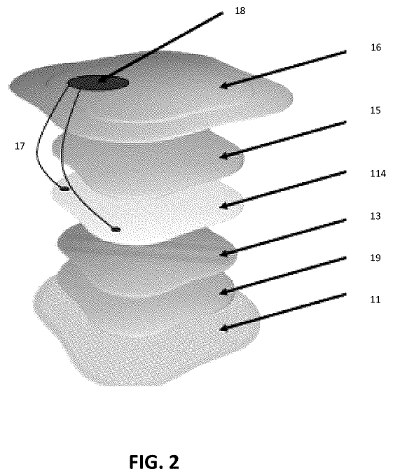

In embodiments, as can be seen in FIG. 2, for example, a bandage 100 may include an electroosmotic pump 114 to pump fluid, such as exudate away from the wound under the bandage to control moisture level at the wound. In embodiments, the bandage 100 includes perforated silicone film 11 provided below the pump 114 and in contact with the use's skin. The perforations allow fluid, or exudate to pass into the bandage 100 from the wound. In embodiments, an adhesive may be provided on a bottom surface of the film 11 such that the film sticks to the user's skin. In embodiments, a nonwoven layer 13 may be provided between the pump 114 and the silicone film 11 and allows fluid to flow therethrough. In embodiments, the nonwoven layer 13 may provide filtering of the liquid or exudate as it passes through to the pump 114. In embodiments, a foam layer 19 may be provided between the silicone film 11 and the nonwoven layer 13 and fluid may pass through the foam layer 19 and into the nonwoven layer 13. In embodiments, the foam layer 19 helps to wick fluid up and away from the film 11 and the wound. In embodiments, the pump 114 may be controlled by electronics module 18. In embodiments, the electronics module 18 may include a power source to provide power to the pump 114. In embodiments, sensor chips may be integrated into electronics module 18 located on the non-patient contacting side of the bandage 100 attached to the polyurethane film 16, for example. In embodiments, data may be communicated from the pump 114 either wirelessly or by insulated wires 17, for example, to the electronics module 18. In embodiments, the layers of the bandage 100 may include contact areas that may be aligned such that they are in contact with each other throughout the bandage. In embodiments, these contact areas may be used to provide power to the pump 114. In embodiments, these contact areas may also be used to provide information to the electronic module 18. In embodiments, data collected may be stored locally in the electronics module 18 and/or may be transmitted wirelessly to a computer, or portable electronic device, for viewing and long-term storage of data. In embodiments, the data collected may be transmitted to and stored in or processed by a remote server that may be associated with a clinician, patient or third party. In embodiments the electronics module 18 may include a processor or other control device to control operation of the pump 114. In embodiments, the electronics module 18 may include a memory operable connected to the processor including processor executable instruction that when executed by the processor provide control signals to the pump 114 to turn activate and deactivate the pump to maintain a desired moisture level at the wound cite. An absorbent layer 15 may be mounted above the pump 114 and liquid may be pumped into the absorbent layer. In embodiments, the pump 114 may be activated based on a timer or based on a moisture level at the pump 114. As noted above, a polyurethane film 16 may be provided above the absorbent layer 15 and the electronics module 18 may be mounted on a top surface of the film 16. The bandage 100 may also include one or more sensors that may be used to provide wound information in substantially the same manner as described above with respect to the bandage 10 to control the electroosmotic pump 114.

Although the present invention has been described in relation to particular embodiments thereof, many other variations and modifications and other uses will become apparent to those skilled in the art. It is preferred, therefore, that the present invention be limited not by the specific disclosure herein.

What is claimed is:

1. A bandage comprising:
a silicone contact layer positioned adjacent a wound and a user's skin when the bandage is in place;
an electroosmotic pump mounted above the silicone contact layer, away from the wound, and operable to selectively draw fluid through the silicone contact layer;
an absorbent layer positioned above the electroosmotic pump and in fluid communication therewith such that the absorbent layer absorbs fluid that is drawn using the electroosmotic pump;
a flexible top layer provided above the absorbent layer; and
an electronic module electrically connected to the electroosmotic pump to selectively activate the electroosmotic pump to draw fluid away from the wound and control a moisture level at the wound.

2. The bandage of claim 1, wherein the silicone contact layer includes a plurality of openings formed therethrough and configure to allow fluid to pass from the wound to the electroosmotic pump.

3. The bandage of claim 1, further comprising an adhesive layer provided between a bottom surface of the silicone contact layer and configured to secure the silicone contact layer to the user's skin.

4. The bandage of claim 2, wherein the electroosmotic pump is in fluid communication with the wound via the plurality of openings in the silicone contact layer.

5. The bandage of claim 1, wherein the electroosmotic pump includes:
an anode positioned adjacent a top surface of the silicone contact layer;
a porous membrane positioned above the anode; and
a cathode provided above the porous membrane and adjacent to the absorbent,
wherein, when a voltage is applied between the anode and the cathode, liquid is drawn upward from the wound, through the silicone contact layer, through the porous membrane and into the absorbent layer.

6. The bandage of claim 5, wherein the electroosmotic pump includes a first bonding layer positioned between the anode and the porous membrane and a second bonding layer positioned between the porous membrane and the cathode.

7. The bandage of claim 1, wherein the electronics module is operably connected to the anode and the cathode and includes a control circuit to apply a voltage between the anode and the cathode.

8. The bandage of claim 1, wherein the control circuit varies the voltage applied between the anode and the cathode to maintain desired moisture level in the wound.

9. The bandage of claim 1, further comprising a sensor provided adjacent the wound and operably connected to the electronics module to provide wound information associated with the wound to control operation of the electroosmotic pump based on the wound information.

10. The bandage of claim 9, wherein the electronics module includes memory and the wound information is stored in the memory.

11. The bandage of claim 9, wherein the electronics module includes a transceiver configured to send and receive information wirelessly and the wound information is sent to a remote electronic device.

12. The bandage of claim 11, wherein control information is received by the transceiver and used to control operation of the electroosmotic pump.

13. The bandage of claim 1 further comprising:
a first cathode electrically connected to the electronic module; and
a first anode electrically connected to the electronic module,
wherein the first cathode and the first anode are positioned in contact with the user's skin and apply a voltage to the wound.

14. A bandage comprising:
a silicone contact layer positioned adjacent a wound and a user's skin when the bandage is in place;
a foam layer positioned above the silicone contact layer such that fluid from the wound passes therethrough;
a nonwoven layer positioned above the foam layer, wherein exudate passes from the foam later through the nonwoven layer and the nonwoven layer filters debris from the exudate;
an electroosmotic pump mounted above the nonwoven layer, away from the wound, and operable to selectively draw fluid through the foam layer, the nonwoven layer and the silicone contact layer;
an absorbent layer positioned above the electroosmotic pump and in fluid communication therewith such that the absorbent layer absorbs fluid that is drawn using the electroosmotic pump;
a flexible top layer provided above the absorbent layer; and
an electronic module electrically connected to the electroosmotic pump to selectively activate the electroosmotic pump to draw fluid away from the wound and control a moisture level at the wound.

15. The bandage of claim 14, wherein the silicone contact layer includes a plurality of openings formed therethrough and configure to allow fluid to pass from the wound to the electroosmotic pump.

16. The bandage of claim 14, further comprising an adhesive layer provided between a bottom surface of the silicone contact layer and configured to secure the silicone contact layer to the user's skin.

17. The bandage of claim 16, wherein the electroosmotic pump is in fluid communication with the wound via the plurality of openings in the silicone contact layer.

18. The bandage of claim 14, wherein the electronics module is operably connected to the electroosmotic pump and includes a control circuit to apply a voltage.

19. The bandage of claim 18, wherein the control circuit varies the voltage to the electroosmotic pump to maintain a desired moisture level in the wound.

20. The bandage of claim 14, wherein the electronics module includes a timer and the electroosmotic pump is controlled based in timing information.

21. The bandage of claim 14, further comprising a sensor positioned at the electroosmotic pump and operable connected to the electronics module, wherein the sensor provides moisture information at the electroosmotic pump and the electroosmotic pump is controlled based on a moisture level at the electroosmotic pump.

22. The bandage of claim 14, further comprising:
a first cathode electrically connected to the electronic module; and
a first anode electrically connected to the electronic module,
wherein the first cathode and the first anode are positioned in contact with the user's skin and apply a voltage to the wound.
